(19) United States
(12) Patent Application Publication
(10) Pub. No.: US 2006/0127387 A1
(43) Pub. Date: Jun. 15, 2006

(54) Compositions based on polysaccharides and protein C and methods of using the same for preventing and treating sepsis and other conditions

(76) Inventors: Bashir A. Zikria, Norwood, NJ (US); J. Dean Zikria, Old greenwich, CT (US)

Correspondence Address: Evelyn M. Sommer, Suite 825, 250 Park Avenue, New York, NY, 10022, US

(21) Appl. No.: 11/280,104

(22) Filed: Nov. 17, 2005

Related U.S. Application Data
(63) Continuation-in-part of application No. 08/837,840, filed on Apr. 22, 1997.

(51) Int. Cl. A61K 38/17; A61K 31/7076; A61K 31/225; A61K 38/44; A61K 31/366; A61K 31/7034

(52) U.S. Cl. 424/94.4; 514/12; 514/54; 514/59; 514/60; 514/47; 514/460; 514/548

(57) ABSTRACT

The present invention provides pharmaceutical compositions useful for the prevention and treatment of sepsis and other conditions such as stroke, reperfusion injury and heart attacks, containing 1) at least one macromolecular polysaccharide selected from the group consisting of hydroxyethyl starch, dextran, glycogen and mixtures thereof and 2) activated protein C. The compositions can further comprise at least one member selected from the group consisting of at least one antioxidant and/or at least one anti-infective. The present invention further provides methods of treating human subjects prior to or during sepsis and other conditions such as stroke, reperfusion injury and heart attacks to prevent leakage of macromolecules from capillary endothelial junctions and simultaneously prevent thrombosis and fibrin formation, reduce inflammation and improve microcirculation by intravenously administering to a subject in need of such treatment an effective amount of the said composition.

The present invention relates to compositions and methods for the prevention and treatment of sepsis, and other conditions such as stroke, reperfusion injury and heart attacks. In a particular aspect, the invention relates to compositions comprising 1) at least one macromolecular polysaccharide selected from the group consisting of hydroxyethyl starch, dextran, glycogen and 2) protein C in a pharmaceutically acceptable liquid carrier therefor. The compositions can further comprise at least one member selected from the group consisting of dehydroascorbic acid, superoxide dismutase, glutathione peroxidase, catalase, hydroxyethyl rutoside, cyclic adenosine monophosphate, vitamin C, hemoglobin, polysaccharide-conjugated hemoglobin, von Willebrand factor, Cerovive, citicoline, poly(ADP-ribose) polymerase inhibitor, oxidant detoxification catalyst, adenosine 2a (A2a) receptor agonist, adenosine 1 (A1) receptor agonist, adenosine, inosine, xanthin oxidase inhibitor, polyethylene-glycol-modified albumin, adenosine triphosphate, histamine, taurine, simvastatin, atrial natriuretic peptide, sphinogosine 1-phosphate, apyrase, secretory leukocyte protease inhibitor, antithrombin III, adrenomedullin, intravenous immunologlobulin, sodium beta-aescin, Δ²-1,2,3-triazoline and aminoalkylpyridine, aromatase inhibitors, and neuropilin-1, edaravone, dimethylthiourea, α-phenyl-N-tert-butyl nitrone, polynitroxyl albumin, phalloidin, amaritin, interleukin-1 receptor antagonist, and the antioxidant subgroup consisting of tocopherols, tocotrienols, carotenoids, minerals and mineral-containing organic compounds, polyphenols, lipoic acids, transition metal ion-binding proteins, melatonin, hormones, polyamines, tamoxifen and its metabolites and propofol. The compositions can further comprise at least one anti-infective. The invention also relates to methods of treating human subjects before and/or during septic attacks, and other conditions such as stroke, reperfusion injury and heart attacks, to prevent leakage of macromolecules from capillary endothelial junctions and simultaneously prevent thrombosis and fibrin formation, reduce inflammation and improve microcirculation, which comprises intravenously administering to a subject in need of such treatment an effective amount of the said composition.

Sepsis is a major cause of morbidity and mortality in humans and animals. It is estimated that more than 700,000 episodes of severe sepsis occur each year (Angus et al. Crit. Care Med. 29:1303-1310 (2001)) and ⅓ of the patients die. Sepsis has become the leading cause of death in the noncoronary intensive care units and the 13^{th} leading cause of death in the United States overall (Sands et al. JAMA 278:234-240 (1997)). It is also the leading cause of death in young livestock, affecting 7.5-29% of neonatal calves (Morris et al. Am. J. Vet. Res. 47:2554-2565 (1986)), and is a common medical problem in neonatal foals (Hoffman et al. J. Vet. Int. Med. 6:89-95 (1992)). Despite the major advances in the treatment of serious infections, the incidence of sepsis continues to increase and is among the leading causes of infectious death. Some factors responsible for the expected increase in incidence of sepsis is an aging population, an increasing immunosuppressed population, increased use of invasive catheters and prosthetic materials, and the growing problem of antimicrobial resistance (Larosa Cleve. Clin. J. Med. 69(1):65-73 (2002)).

In 1991, a consensus panel of the American College of Chest Physicians and the Society of Critical Care Medicine defined sepsis related diseases (Crit. Care Med. 20:864-874 (1992)). Systemic Inflammatory Response Syndrome (SIRS) is the host response to critical illness of either infectious or noninfectious origin. Examples of noninfectious causes include burns, trauma, and pancreatitis. SIRS can be readily diagnosed by the presence of at least two of the following four signs: hyperthermia or hypothermia, tachycardia, tachypnea, leukocytosis or leukopenia. Sepsis is defined as SIRS due to a presumed or known infection. Severe sepsis is sepsis with an acute associated organ failure. Septic shock is a subset of severe sepsis, defined as persistently low mean arterial blood pressure despite adequate fluid resuscitation. Refractory septic shock is persistently low mean arterial blood pressure despite vasopressor therapy and adequate fluid resuscitation.

Severe sepsis can start with an infection in any part of the body and the pathogens can be bacteria (either Gram negative or Gram positive), fungi, viruses, and parasites. Sepsis is also commonly caused by trauma, burns, difficult newborn deliveries, organ transplantation, and intestinal torsion (especially in dogs and horses). The systemic invasion of microorganisms presents two distinct problems. First, the growth of the pathogen can directly damage tissues, organs and vascular function. Second, toxic components of the microorganisms can lead to rapid systemic inflammatory responses that can quickly damage vital organs and lead to circulatory collapse (septic shock). Many patients who enter septic shock die. The effect by endotoxin is manifested by its binding to cells such as monocytes/macrophages or endothelial cells, and triggering them to produce various mediators such as tumor necrosis factor alpha, adhesive molecules and various interleukins. These cytokines have a direct toxic effect on tissues. They also promote nitric oxide synthase activity, tissue infiltration by neutrophils, and neutrophil activity. In addition, they activate phospholipase A₂, leading to increased concentration of platelet-activating factor and promote coagulation in multiple ways. Activation of the coagulation system results in thrombin generation and platelet thrombi formation in the microcirculation in many tissues. The pathogenesis of this syndrome involves the activation of the intrinsic coagulation system by factor XII. Activated factor XII initiates the intrinsic coagulation cascade and eventually fibrinogen is converted to fibrin and clotting occurs. Uncontrolled activation of coagulation, usually accompanied by shock, will result in thrombosis and consumption of clotting factors II, V, and VIII. Some common complications of disseminated intravascular coagulation are severe clinical bleeding, thrombosis, tissue ischemia and necrosis, haemolysis and organ failure.

Sepsis is a very complicated disease and many methods for prevention and/or treating it have been proposed by focusing on certain aspects of the disease. Some of them focus on the alleviation of infections using, for example, certain anti-infectives to prevent and treat the disease at its source. More focus on preventing or alleviating the damages caused directly by the infection and indirectly by the human immunal system in response to the infections, such as using agents that block inflammatory mediators associated with the pathophysiology of sepsis. Yet some others attempt to use a combination of therapeutics for battling the disease at both fronts.

U.S. Pat. No. 6,660,267 teaches therapeutic compositions and methods for preventing and treating blood-borne and toxin mediated diseases in humans and animals and the composition mainly comprises antibody-antibiotic conjugates. U.S. Pat. No. 6,673,346 teaches compositions comprising anti-C5a antibodies and a method for the prevention and treatment of sepsis. U.S. Pat. No. 4,772,465 teaches the use of ciprofloxacin and psedudomonas immune globulin for treating polymicrobial burn wound sepsis. U.S. Pat. No. 5,660,826 teaches the use of an antagonist to parathyroid hormone related protein for the prophylaxis of sepsis. U.S. Pat. No. 5,998,482 teaches the use of synthetic polycationic amphiphilic substances with fatty acid or hydrocarbon substitutes as anti-sepsis agents. U.S. Pat. No. 6,624,140 teaches synthetic peptides with antimicrobial and endotoxin neutralizing properties for management of the sepsis syndrome. U.S. Pat. No. 6,534,648 teaches the use of isolated algal lipopolysaccharides for inhibiting endotoxin-initiated sepsis. U.S. Pat. No. 6,042,821 teaches the method for treating sepsis with chemokines. U.S. Pat. No. 6,063,764 teaches the method for prophylactically or therapeutically treating sepsis or septic shock using an inhibitor to tissue factor. U.S. Pat. No. 6,406,688 teaches the method for treating sepsis using chamohine beta-10, alone or in conjunction with an anti-infective. U.S. Pat. No. 6,315,999 teaches the use of an antibody to tumor necrosis factor-alpha and an antibody to bacterial lipopolysaccharide. U.S. Pat. No. 5,714,469 teaches the use of a specific peptide alone or in conjunction with an anti-infective for the treatment of sepsis. U.S. Pat. No. 5,993,811 teaches the use of an antibody reactive to procalcitonin for the treatment of sepsis. U.S. Pat. No. 5,756,481 teaches the use of cysteine for treating sepsis.

Of particular relevance to the instant invention, is the use of activated protein C alone or in conjunction with other therapeutic agents for the treatment of sepsis.

U.S. Pat. No. 6,344,197 teaches methods for treating sepsis using a combination therapy of protein C (with its anti-coagulant/anti-inflammatory properties) and BPI protein (with its bactericidal and endotoxin neutralizing activities).

U.S. Pat. No. 5,093,117 teaches compositions and methods for the treatment or prophylaxis of sepsis or septic shock using a bactericidal effective amount of polyclonal immunoglobublins and a blood clot-dissolving effective amount of protein C.

U.S. Pat. No. 4,775,624; 5,270,040 and 5,681,932 (assigned to Eli Lilly and Co.) teach the vectors and compounds for expression of human protein C. EP 0662513A1 (assigned to Eli Lilly and Co.) teaches the methods to prevent or minimize autodegradation of activated protein C.

Activated protein C, a vitamin K dependent protein of blood plasma, is a protein of major physiological importance, especially for its anticoagulant, anti-inflammatory and profibrinolytic properties. In consort with other proteins, protein C functions as perhaps the most important down-regulator of blood coagulation resulting in thrombosis. In other words, the protein C enzyme system represents a major physiological mechanism for anticoagulation. An important cofactor for activated protein C is protein S, another vitamin K dependent plasma protein and protein S substantially increases activated protein C-mediated hydrolysis of factors Va and VIIIa.

In 2001, drotrecogin alpha (activated), a recombinant form of human Activated Protein C, under the trademark Xigris® (Eli Lilly), was approved by the US FDA for the treatment of severe sepsis and it is indicated for the reduction of mortality in adult patients with severe sepsis who have a high risk of death.

Drotrecogin alfa (activated) is a serine protease with the same amino acid sequence as human plasma-derived Activated Protein C. Drotrecogin alfa (activated) is a glycoprotein of approximately 55 kilodalton molecular weight, consisting of a heavy chain and a light chain linked by a disulfide bond. Drotrecogin alfa (activated) and human plasma-derived Activated Protein C have the same sites of glycosylation, although some differences in the glycosylation structures exist. An established human cell line possessing the complementary DNA for the inactive human Protein C zymogen secretes the protein into the fermentation medium. Human Protein C is enzymatically activated by cleavage with thrombin and subsequently purified.

Activated Protein C exerts an antithrombotic effect by inhibiting Factors Va and VIIIa (Esmon et al. Thromb. Haemost. 82:251-258 (1999)). In vitro data indicate that Activated Protein C has indirect profibrinolytic activity through its ability to inhibit plasminogen activator inhibitor-1 (PAI-1) and limiting generation of activated thrombin-activatable-fibrinolysis-inhibitor. Additionally, in vitro data indicate that Activated protein C may exert an anti-inflammatory effect by inhibiting human tumor necrosis factor production by monocytes, by blocking leukocyte adhesion to selecting, and by limiting the thrombin-induced inflammatory responses within the microvascular endothelium.

Bleeding is the most common serious adverse effect associated with Xigris therapy and Xigris is contraindicated in patients with the clinical situations in which bleeding could be associated with a high risk of death or significant morbidity.

While Xigris proves effective among some patients with severe sepsis, it is not intended to prevent or reverse all of the harmful effects of sepsis.

Capillary leak is another serious aspect of sepsis. During the systemic inflammatory response, the inflammatory mediators initiate a biochemical chain of events that increase capillary permeability and deteriorate capillary membrane stability. These mediators include pharmacologically active amines such as histamine and 5-hydroxytryptamine, polypeptides such as bradykinin, kallikrein and leukotoxine, the prostaglandins, and various complements including derivatives thereof. These mediators act specifically on the junction of the endothelial cells of capillaries so that the junctions cannot contain colloids such as serum albumin within the vessel. The serum albumin escapes into the interstitium creating a nonfunctional “third space”, the volume of which increases proportionally to albumin leakage and the presence of cytokines as well as proteolytic enzyme activity within the matrix. This leakage further widens capillary membrane-transport between the circulatory system and the functional cells resulting in cellular anoxia, cellular energy deficit, acidosis and possibly leads to sequential organ failure. These effects, in combination with ischemia due to blood clotting from thrombosis (which Xigris intends to alleviate) and the inability to extract oxygen are believed to be among the major factors responsible for multiple organ failures in severe sepsis.

In the past, the problem of albumin leakage and the concurrent creation of a third space has been approached through pharmacological means. The same inventors (U.S. Pat. No. 6,207,654) approached the problem as a biophysical phenomenon by utilizing natural or synthetic polysaccharides (macromolecules) as sealants and capillary membrane stabilizers to prevent or substantially reduce the escape of albumin and other molecules through the junction of the endothelial cells of the capillaries as well as stabilize the colloidal oncotic pressure. In the '654 invention, polysaccharides were used in combination with a member selected from the group of interferon and interleukin for treating human subjects to prevent leakage of macromolecules from capillary endothelial junctions as a consequence of severe life threatening side effects due to various diseases including septic shock.

In addition to capillary leak, thrombosis, fibrin formation and inflammation, oxidative damages also play a role in sepsis. When clotting factors run out, the patient develops a bleeding tendency, and thrombosed areas may undergo reperfusion which may further organ injury due to the development of oxygen free radicals. U.S. Pat. No. 5,198,212 teaches a method and compositions for the treatment of trauma-associated sepsis using gamma interferon alone or in combination with an antibiotic. The invention was partially based on the discovery that endotoxin induces oxidative lung injury and lipid peroxidation and that catalase treatment attenuates the harmful effects (Taylor et al. J. Clin. Invest. 79:918-925 (1987)). U.S. Pat. No. 5,354,771 teaches methods for treatment of free-radical-mediated tissue injury, particularly sepsis using branch chain amino acids.

In copending application Ser. No. 08/837840, the same inventors have used such polysaccharides in combination with an antioxidant for treating human subjects to prevent leakage of macromolecules from capillary endothelial junctions and simultaneously preventing damage to the capillaries and surrounding tissues due to the presence of released free radicals, due to various diseases including septic shock.

The inventors have found a composition comprising at least one macromolecular polysaccharide selected from the group consisting of hetastarch (HES), dextran and glycogen, and activated protein C can be used to treat patients with severe sepsis. This composition can prevent leakage of macromolecules from capillary endothelial junctions and simultaneously prevent thrombosis and fibrin formation, reduce inflammation and improve microcirculation. The inventors have also found out that a composition comprising at least one antioxidant and/or at least one anti-infective, in addition to the above mentioned macromolecular polysaccharide and activated protein C can confer more beneficial effects on the patients with severe sepsis.

Xigris is supplied commercially as a sterile, lyophilized, and white to off-white powder for intravenous infusion. Although the recombinant human activated protein C (Xigris) is preferred for this invention, the protein C that can be used for this composition and method may also include other species or derivatives having protein C proteolytic, amidolytic, esterolytic and biological (anticoagulant, pro-fibrinolytic, and anti-inflammatory) activities. Examples of protein C derivatives are described in U.S. Pat. No. 5,453,373 and 5,516,650.

Hydroxyethyl starch (Hespan® U.S. Pat. No. 3,523,938) is an artificial colloid derived from a waxy starch, composed almost entirely of amylopectin. The branched amylopectin polymer has a degree of polymerization on the order of several hundred glucose residues. The segments between the branched points average about 25 glucose residues linked by alpha-D-(1-4) glucosidic bonds, while the branched points are linked by alpha-D-(1-6) bonds. Hydroxyethyl ether groups are introduced into the glucose units of the starch and the resultant material is hydrolyzed. Clinical hetastarch is characterized by its molecular weight and its degree of substitution. The average molecular weight is approximately 480,000 Daltons with a range of 400,000 to 500,000 and with 80% of its polymer units falling within the range of 30,000 to 2,400,000 Daltons. The molar substitution is 0.7 which means hetastarch has 7 hydroxyethyl groups for every 10 glucose units. The polymerized glucose units in hetastarch are joined primarily 1-4 linkage with hydroxethyl groups being attached primarily at the number 2 position. The polymer closely resembles glycogen. The degree of branching is approximately 1:20 which means that there is one 1-6 branch for every 20 glucose units. The chemical name for hetastarch is hydroxyethyl starch. The structural formula is as follows: 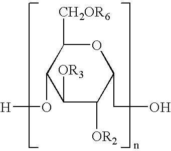
amylopectin derivative in which R₂, R₃, and R₆ are H or CH₂CH₂OH, or R₆ is a branching point in the starch polymer connected through a 1-6 linkage to additional alpha-D-glucopyranosyl units.

The colloidal properties of 6% hetastarch approximate those of human albumin. Intravenous infusion of HES results in expansion of the plasma volume slightly in excess of the volume infused but which decreases over the succeeding 24-36 hours. This expansion of plasma volume improves the hemodynamic status of the subject for 24 hours or longer. Hydroxyethyl starch molecules below 50,000 Daltons are rapidly eliminated by renal excretion with approximately 40% of a given total dose appearing in the urine in 24 hours. The hydroxethyl group is not cleaved by the body, but remains intact and attached to the glucose units when excreted. Significant quantities of glucose are not produced (metabolism) as hydroxyethylation prevents complete metabolism. Despite its extensive clinical use hetastarch has not been observed to act in a way more than merely exerting a colloidal oncotic pressure when compared to albumin.

Hydroxyethyl starch is administered by intravenous infusion only. In adults the amount usually administered is 500 to 1500 mls. Doses of 1500 mls per day of 6% hydroxyethyl starch per 70 kg man have been used in postoperative open heart operations and trauma patients. Hydroxyethyl starch can be delivered in 0.9% saline, 5% dextrose or Ringer's lactate.

The inventors have utilized other polysaccharides in addition to hetastarch and have produced promising results. These polysaccharide macromolecules include glycogen and dextran.

Glycogen is a readily mobilized storage form of glucose. It is a very large polymer of glucose residues. Most of the glucose residues are linked by alpha- 1-4 glycosidic bonds of which there is one in about 10 residues. Glycogen granules are 100 to 400 Angstrom and have a molecular weight range of about 270,000 to 350,000 Daltons. The molecules of glycogen do not have a unique size. The average molecular weight is several hundred kilodaltons.

Dextran, another polysaccharide is made up of glucose residues only, mainly in alpha-1-6 linkage. Occasional branches are formed by alpha-1-2, alpha-1-3 or alpha-1-4 linkages. The nature of the linkages are dependent on the source of the dextran. Certain bacteria secrete dextran as a by-product of their growth and commercial dextran is manufactured by bacterial culture procedures. By varying the growth conditions of the bacteria, the molecular weight of the dextran can be controlled to bring about the desired size. Useful molecular weights for plasma substitution range from 100,000 to 500,000. Dextran of appropriate molecular size does not pass through the capillary pores and therefore, can replace plasma proteins as colloid osmotic agents.

Hydroxyethyl starch, dextran and glycogens can be used singly or in combination, such as the mixture of hydroxyethyl starch and dextran.

The total molecular weight range and composition of the invention may differ due to the fact that these macromolecules characteristically exhibit a wide range of molecular weights. The molecular weight ranges of the macromolecules used may also be varied depending on the specific clinical application.

It is critical that the compositions contain the macromolecules in a molecular size and concentration to effectively seal the capillary junctions and stabilize the capillary membranes. The sealant effect is accomplished by a biophysical process due to the adhesiveness and configuration of the macromolecules, and because of their size.

Few toxic reactions have been observed when using either dextran or hetastarch for fluid replacement therapy.

The applicants have prepared the following compositions of polysaccharide macromolecules and activated protein C and utilized them in order to inhibit or prevent capillary leakage and reduce inflammation for the prevention and treatment of sepsis. The procedures carried out by the applicants have shown the following: reduction of the inflammatory reaction, reduction of capillary leakage, prevention or reduction of thrombosis and fibrin formation, and improvement in microcirculation.

The applicants have further prepared the compositions containing at least one agent, in particular antioxidant, in addition to polysaccharide macromolecules and protein C. Examples include dehydroascorbic acid, superoxide dismutase, glutathione peroxidase, catalase, hydroxyethyl rutoside, cyclic adenosine monophophate, vitamin C, hemoglobin, polysaccharide-conjugated hemoglobin, von Willebrand factor, Cerovive, citicoline, poly(ADP-ribose) polymerase inhibitor, oxidant detoxification catalyst, adenosine 2a (A2a) receptor agonist, adenosine 1 (A1) receptor agonist, adenosine, inosine, xanthin oxidase inhibitor, polyethylene-glycol-modified albumin, adenosine triphosphate, histamine, taurine, simvastatin, atrial natriuretic peptide, sphinogosine 1-phosphate, apyrase, secretory leukocyte protease inhibitor, antithrombin III, adrenomedullin, intravenous immunologlobulin, sodium beta-aescin, Δ²-1,2,3-triazoline and aminoalkylpyridine, aromatase inhibitors, and neuropilin-1, edaravone, dimethylthiourea, α-phenyl-N-tert-butyl nitrone, polynitroxyl albumin, phalloidin, amaritin, interleukin-1 receptor antagonist and the antioxidant subgroup consisting of tocopherols, tocotrienols, carotenoids, minerals and mineral-containing organic compounds, polyphenols, lipoic acids, transition metal ion-binding proteins, melatonin, hormones, polyamines, tamoxifen and its metabolites and propofol. The procedures carried out by the applicants have shown the additional benefit of the reduction of damage to endothelial cells and other tissues.

It is also within the spirit of this invention to further include at least one anti-infective to the final composition for maximum strength in preventing and treating sepsis.

In addition to sepsis, the compositions of this invention have also been indicated for other conditions, including stroke, reperfusion injury and heart attacks.

The following compositions are given by way of example to illustrate the representative formulations suitable for the indicated conditions.

### COMPOSITIONS

HES and Activated Protein C.
Glycogen and Activated Protein C.
Dextran and Activated Protein C.
HES, Vitamin C and Activated Protein C.
Glycogen, Vitamin C and Activated Protein C.
Dextran, Vitamin C and Activated Protein C.
HES, catalase, and Activated Protein C.
Glycogen, catalase, and Activated Protein C.
Dextran, catalase, and Activated Protein C.
HES, superoxide dismutase and Activated Protein C.
Glycogen, superoxide dismutase and Activated Protein C.
Dextran, superoxide dismutase and Activated Protein C.

The compositions are prepared using 5-15% HES, 5-15% glycogen or 5-15% dextran dependent on the clinical indications. When using two polysaccharides they can be used in a ratio of from 4:1 to 1:4. The compositions are always introduced intravenously. Treatment can be repeated as indicated.

The preferred dosage of Activated Protein C is 24 micrograms per kilogram of patient body weight per hour for 96 hours of infusion. The maximum duration of infusion of Activated Protein C from one preparation step is 12 hours. Therefore, multiple infusion periods will be needed to cover the entire 96 hour duration of administration.

It is possible in accordance with the invention to administer the polysaccharide (or mixtures of polysaccharides) and with activated protein C one after another or to administer them simultaneously. The antioxidant and/or the anti-infective, when desired, can be administered simultaneously with the other active compounds or separately.

The storage and delivery methods of the compositions of the invention can be any of the following,
1. Standard Mixture Delivery, wherein the polysaccharides (HES, and/or dextran, and/or glycogen) are in a mixture with the protective agents in a solution within an I.V. bag for storage and administration to a patient,
2. Piggyback Delivery, wherein the solution containing polysaccharides and the solution containing protective agents are in different compartments of the same I.V. bag and will only mix during administration when the I.V. bag is connected to the reservoir where each portion drip into after leaving their respective compartments in the bag. The mixed solution then flows from the reservoir into the bloodstream of the patient, and
3. Just-in-time Combination Delivery, wherein the polysaccharides and the protective agents are in the same I.V. bag, but in separate compartments isolated by a separating means (such as a seal, a rupturable membrane or any other known means) and will only mix during administration when the separating means is disabled between the two compartments, allowing the polysaccharides and the protective agents to become a mixture before exiting the I.V. bag.

While the compositions disclosed in this invention can be used after the onset of the diseases for treatment, they can also be used prophylactically to prevent the diseases from taking place or reducing the severity of the diseases at onsets.

We claim:

1. Method of treating human subjects to prevent leakage of macromolecules from capillary endothelial junctions and simultaneously prevent thrombosis and fibrin formation, reduce inflammation and improve microcirculation which comprises intravenously administering to a subject in need of such treatment an effective amount of composition comprising 1) at least one macromolecular polysaccharide selected from the group consisting of hydroxyethyl starch, dextran, glycogen and mixtures thereof and 2) activated protein C in a pharmaceutically acceptable liquid carrier therefor.

2. Method according to claim 1 wherein said activated protein C is recombinant human activated protein C.

3. Method according to claim 1 wherein said composition additionally contains at least one member selected from the group consisting of dehydroascorbic acid, superoxide dismutase, glutathione peroxidase, catalase, hydroxyethyl rutoside, cyclic adenosine monophosphate, vitamin C, hemoglobin, polysaccharide-conjugated hemoglobin, von Willebrand factor, Cerovive, citicoline, poly(ADP-ribose) polymerase inhibitor, oxidant detoxification catalyst, adenosine 2a (A2a) receptor agonist, adenosine 1 (A1) receptor agonist, adenosine, inosine, xanthin oxidase inhibitor, polyethylene-glycol-modified albumin, adenosine triphosphate, histamine, taurine, simvastatin, atrial natriuretic peptide, sphinogosine 1-phosphate, apyrase, secretory leukocyte protease inhibitor, antithrombin III, adrenomedullin, intravenous immunologlobulin, sodium beta-aescin, Δ²-1,2,3-triazoline and aminoalkylpyridine, aromatase inhibitors, and neuropilin-1, edaravone, dimethylthiourea, α-phenyl-N-tert-butyl nitrone, polynitroxyl albumin, phalloidin, amaritin, interleukin-1 receptor antagonist, and the antioxidant subgroup consisting of tocopherols, tocotrienols, carotenoids, minerals and mineral-containing organic compounds, polyphenols, lipoic acids, transition metal ion-binding proteins, melatonin, hormones, polyamines, tamoxifen and its metabolites and propofol.

4. Method according to claim 1 wherein said composition additionally contains at least one anti-infective.

5. A composition comprising a member selected from the group consisting of hydroxyethyl starch, dextran, glycogen and mixtures thereof, and activated protein C.

6. A composition according to claim 5 wherein said protein C is recombinant human activated protein C.

7. A composition according to claim 5 additionally containing at least one member selected from the group consisting of dehydroascorbic acid, superoxide dismutase, glutathione peroxidase, catalase, hydroxyethyl rutoside, cyclic adenosine monophosphate, vitamin C, hemoglobin, polysaccharide-conjugated hemoglobin, von Willebrand factor, Cerovive, citicoline, poly(ADP-ribose) polymerase inhibitor, oxidant detoxification catalyst, adenosine 2a (A2a) receptor agonist, adenosine 1 (A1) receptor agonist, adenosine, inosine, xanthin oxidase inhibitor, polyethylene-glycol-modified albumin, adenosine triphosphate, histamine, taurine, simvastatin, atrial natriuretic peptide, sphinogosine 1-phosphate, apyrase, secretory leukocyte protease inhibitor, antithrombin III, adrenomedullin, intravenous immunologlobulin, sodium beta-aescin, Δ²-1,2,3-triazoline and aminoalkylpyridine, aromatase inhibitors, and neuropilin-1, edaravone, dimethylthiourea, α-phenyl-N-tert-butyl nitrone, polynitroxyl albumin, phalloidin, amaritin, interleukin-1 receptor antagonist, and the antioxidant subgroup consisting of tocopherols, tocotrienols, carotenoids, minerals and mineral-containing organic compounds, polyphenols, lipoic acids, transition metal ion-binding proteins, melatonin, hormones, polyamines, tamoxifen and its metabolites and propofol.

8. A composition according to claim 5 additionally containing at least one anti-infective.
